(19) United States
(12) Patent Application Publication
(10) Pub. No.: US 2008/0269281 A1
(43) Pub. Date: Oct. 30, 2008

(54) Novel Beta-Agonists, Method for Producing Them and Their Use as Drugs

(76) Inventors: Thomas Trieselmann, Warthausen (DE); Rainer Walter, Biberach (DE); Matthew R. Netherton, Danbury, CT (US); Bradford S. Hamilton, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/091,828

(22) PCT Filed: Oct. 27, 2006
(86) PCT No.: PCT/EP06/67875

§ 371 Date: Jun. 20, 2008

(30) Foreign Application Priority Data
Oct. 28, 2005 (DE) 10-2005-052-103.7

(51) Int. Cl. A61K 31/437; C07D 471/04; A61P 3/10; A61P 3/04

(52) U.S. Cl. 514/303; 546/118

(57) ABSTRACT

Compounds of the formula

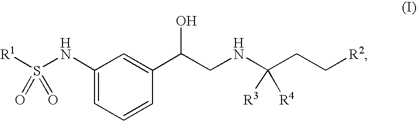

which are selective beta-3-agonists and useful for the treatment of obesity and type II diabetes. Exemplary compounds are:
ethyl 3-{3-[(R)-2-(3-benzenesulphonylamino-phenyl)-2-hydroxy-ethylamino]-3-methyl-butyl}-3H-imidazo[4,5-b]pyridine-5-carboxylate and
3-{3-[(R)-2-(3-benzenesulphonylamino-phenyl)-2-hydroxy-ethylamino]-3-methyl-butyl}-3H-imidazo[4,5-b]pyridine-5-carboxylic acid.

The present invention relates to new beta-agonists of general formula (I)

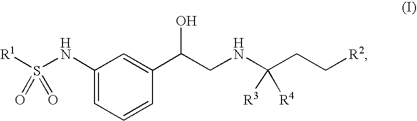

wherein the groups R¹ to R⁴ have the meanings given in the claims and specification, the tautomers, racemates, enantiomers, diastereomers, solvates, hydrates thereof, mixtures thereof and the salts thereof, particularly the physiologically acceptable salts thereof with inorganic or organic acids or bases, processes for preparing these compounds and their use as medicaments.

### BACKGROUND TO THE INVENTION

The treatment of type II diabetes and obesity is based primarily on reducing calorie intake and increasing physical activity. These methods are seldom successful in the long term.

It is known that beta-3 receptor agonists exhibit a significant effect on lipolysis, thermogenesis and the serum glucose level in animal models of type II diabetes (Arch J R. beta(3)-Adrenoceptor agonists: potential, pitfalls and progress, Eur J. Pharmacol. 2002 Apr. 12; 440(2-3):99-107).

Compounds that are structurally similar to the compounds according to the invention and their broncholytic, spasmolytic and antiallergic activity were disclosed for example in DE 2833140.

The aim of the present invention is to provide selective beta-3-agonists which are suitable for preparing medicaments for the treatment of obesity and type II diabetes.

### DETAILED DESCRIPTION OF THE INVENTION

Surprisingly it has been found that compounds of general formula (I) wherein the groups R¹ to R⁴ have the meanings given below act as selective beta-3-agonists. Thus the compounds according to the invention may be used for the treatment of ailments connected with the stimulation of beta-3-receptors.

The present invention therefore relates to compounds of general formula (I)

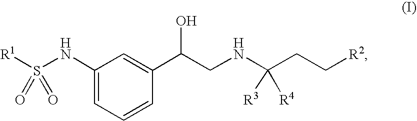

wherein
R¹ denotes a C₁₋₄-alkyl, thienyl, pyridyl or phenyl group,
wherein the phenyl group may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different,
   R² denotes a benzimidazolyl group wherein a methyne group is replaced in the benzyl moiety by a nitrogen atom and
which may additionally be substituted by a fluorine, chlorine or bromine atom, a cyano group or a C₁₋₃-alkyl, carboxy, C₁₋₄-alkyloxy-carbonyl or amino group,
   and R³ and R⁴, which may be identical or different, each denote a C₁₋₃-alkyl group,
   while the alkyl groups contained in the above-mentioned groups may be straight-chain or branched,
   optionally in the form of the tautomers, racemates, enantiomers, diastereomers, solvates, hydrates and mixtures thereof, and optionally the prodrugs, double prodrugs and salts thereof, particularly the physiologically acceptable salts thereof with inorganic or organic acids or bases.

Preferably compounds of general formula (I) are those wherein

R¹ denotes a phenyl group,
R² denotes a benzimidazol-1-yl group wherein a methyne group in the benzyl moiety is replaced by a nitrogen atom and
which is additionally substituted by a carboxy or C₁₋₄-alkyloxy-carbonyl group,
   and R³ and R⁴ in each case represent a methyl group,
   the tautomers, the enantiomers, the diastereomers, the mixtures thereof and the salts thereof.

A preferred sub-group relates to the (R)-enantiomer of the compounds according to the invention of general formula (Ia)

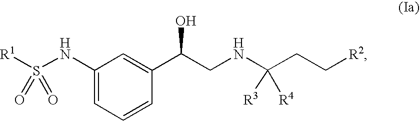

wherein R¹ to R⁴ are as hereinbefore defined.

A preferred sub-group relates to the (S)-enantiomer of the compounds according to the invention of general formula (Ib)

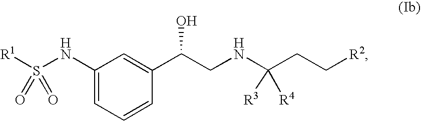

wherein R¹ to R⁴ are as hereinbefore defined.

The following compounds are particularly preferred:
ethyl 3-{3-[(R)-2-(3-benzenesulphonylamino-phenyl)-2-hydroxy-ethylamino]-3-methyl-butyl}-3H-imidazo[4,5-b]pyridine-5-carboxylate,
3-{3-[(R)-2-(3-benzenesulphonylamino-phenyl)-2-hydroxy-ethylamino]-3-methyl-butyl}-3H-imidazo[4,5-b]pyridine-5-carboxylic acid,
ethyl 1-{3-[2-(3-benzenesuphonylamino-phenyl)-2-hydroxy-ethylamino]-3-methyl-butyl}-1H-imidazo[4,5-c]pyridine-6-carboxylate,
1-{3-[2-(3-benzenesulphonylamino-phenyl)-2-hydroxy-ethylamino]-3-methyl-butyl}-1H-imidazo[4,5-c]pyridine-6-carboxylic acid,
ethyl 1-{3-[2-(3-benzenesuphonylamino-phenyl)-2-hydroxy-ethylamino]-3-methyl-butyl}-1H-imidazo[4,5-b]pyridine-5-carboxylate,
1-{3-[2-(3-benzenesulphonylamino-phenyl)-2-hydroxy-ethylamino]-3-methyl-butyl}-1H-imidazo[4,5-b]pyridine-5-carboxylic acid,
ethyl 3-{3-[(R)-2-(3-benzenesulphonylamino-phenyl)-2-hydroxy-ethylamino]-3-methyl-butyl}-3H-imidazo[4,5-c]pyridine-6-carboxylate and
3-{3-[2-(3-benzenesulphonylamino-phenyl)-2-hydroxy-ethylamino]-3-methyl-butyl}-3H-imidazo[4,5-c]pyridine-6-carboxylic acid,
   and the enantiomers and salts thereof.

The invention further relates to compounds of general formula (I) for use as pharmaceutical compositions.

The invention further relates to compounds of general formula (I) for use as pharmaceutical compositions with a selective beta-3-agonistic activity.

The invention further relates to compounds of general formula (I) for preparing a pharmaceutical composition for the treatment and/or prevention of diseases which are associated with the stimulation of beta-3-receptors.

The invention further relates to a method for the treatment and/or prevention of diseases which are associated with the stimulation of beta-3-receptors, by administering to a patient an effective amount of a compound of general formula I.

The invention further relates to a pharmaceutical composition, containing as active substance one or more compounds of general formula (I) optionally in combination with conventional excipients and/or carriers.

The invention further relates to a pharmaceutical composition containing as active substance one or more compounds of general formula (I) or the physiologically acceptable salts thereof and one or more active substances selected from among the antidiabetic agents, inhibitors of protein tyrosine phosphatase 1, substances that influence deregulated glucose production in the liver, lipid lowering agents, cholesterol absorption inhibitors, HDL-raising compounds, active substances for the treatment of obesity and modulators or stimulators of the adrenergic system through alpha 1 and alpha 2 as well as beta 1, beta 2 and beta 3 receptors.

The invention further relates to a process for preparing a compound of general formula (I)

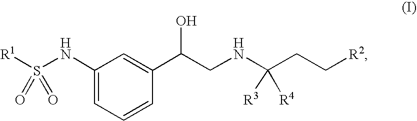

wherein
R¹ to R⁴ may have the meanings given above,
wherein a compound of general formula (II)

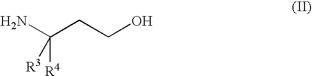

wherein
R³ and R⁴ have the meaning given above may,
is converted by means of a chlorinating agent into a compound of formula (III)

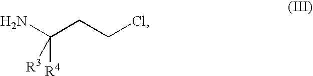

the compound of formula (III), optionally provided with an amino protective group, is reacted with a compound of formula (IV),

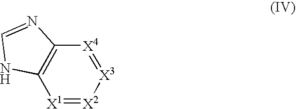

wherein one of the groups X₁ to X₄ denotes a nitrogen atom and the other three groups X₁ to X₄ each represent a —CH═ group, and wherein the compound of formula (IV) may additionally be substituted by a fluorine, chlorine or bromine atom or a C₁₋₃-alkyl, carboxy, C₁₋₄-alkyloxy-carbonyl or amino group,
and the product of formula (V)

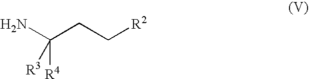

wherein R², R³ and R⁴ have the meaning given above,
is reacted with a compound of formula (VIa), (VIb) or (VIc)

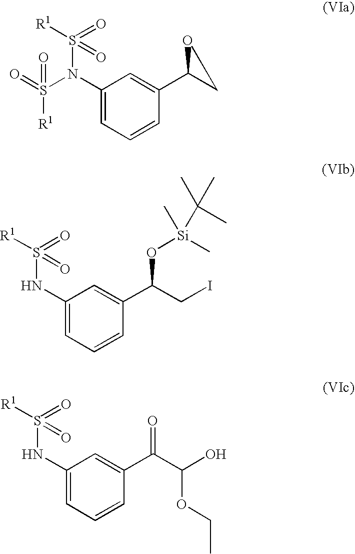

where R¹ has the meaning given above,
and then a desilylation, desulphonation or separation of enantiomers is optionally carried out.

The reaction with the compound (VIc) leads to the racemate, whereas the synthesis with the compounds (VIa) or (VIb) yields the respective (R)-enantiomer. An analogous reaction with the enantiomer of (VIa) or (VIb), leading to the (S)-enantiomer, is naturally also possible.

By alkyl groups, as well as alkyl groups, which are a part of other groups, are meant, unless stated otherwise, branched and unbranched alkyl groups with 1 to 10 carbon atoms, while groups with 1 to 6 carbon atoms are preferred. Particularly preferred are alkyl groups with 1 to 4 carbon atoms, particularly those with 1 or 2 carbon atoms. Examples include: methyl, ethyl, propyl, butyl, pentyl, hexyl, heptyl, octyl, nonyl and decyl. Unless stated otherwise, the above-mentioned terms propyl, butyl, pentyl, hexyl, heptyl, octyl, nonyl and decyl include all the possible isomeric forms. For example the term propyl includes the two isomeric groups n-propyl and iso-propyl, the term butyl includes n-butyl, iso-butyl, sec. butyl and tert.-butyl, the term pentyl includes isopentyl, neopentyl etc.

In the above-mentioned alkyl groups one or more hydrogen atoms may optionally be replaced by other groups. For example these alkyl groups may be substituted by the halogen atoms fluorine, chlorine, bromine or iodine. The substituents are preferably fluorine or chlorine. The substituent fluorine is particularly preferred. If desired all the hydrogen atoms of the alkyl group may be replaced.

Similarly, in the above-mentioned alkyl groups, unless stated otherwise, one or more hydrogen atoms may optionally be replaced for example by OH, NO₂, CN or an optionally substituted group selected from among —O—(C₁-C₅-alkyl), preferably methoxy or ethoxy, —O—(C₆-C₁₄-aryl), preferably phenyloxy, —O-heteroaryl, preferably —O-thienyl, —O-thiazolyl, —O-imidazolyl, —O-pyridyl, —O-pyrimidyl or —O-pyrazinyl, saturated or unsaturated —O-heterocycloalkyl, preferably —O-pyrazolyl, —O-pyrrolidinyl, —O-piperidinyl, —O-piperazinyl or —O-tetrahydro-oxazinyl, C₆-C₁₄-aryl, preferably phenyl, heteroaryl, preferably thienyl, thiazolyl, imidazolyl, pyridyl, pyrimidyl or pyrazinyl, saturated or unsaturated heterocycloalkyl, preferably pyrazolyl, pyrrolidinyl, piperidinyl, piperazinyl or tetrahydro-oxazinyl, an amine group, preferably methylamine, benzylamine, phenylamine or heteroarylamine, saturated or unsaturated bicyclic ring systems, preferably benzimidazolyl and C₃-C₈-cycloalkyl, preferably cyclohexyl or cyclopropyl.

By alkenyl groups, as well as alkenyl groups which are a part of other groups, are meant branched and unbranched alkyl groups with 1 to 10 carbon atoms, preferably 1 to 6, particularly preferably 1 to 4 carbon atoms, which contain at least one carbon-carbon double bond. Examples include: ethenyl, propenyl, methylpropenyl, butenyl, pentenyl, hexenyl, heptenyl, methylheptenyl, octenyl, nonenyl and decenyl. Unless stated otherwise, the above-mentioned terms propenyl, butenyl, pentenyl, hexenyl, heptenyl, octenyl, nonenyl and decenyl include all the possible isomeric forms. For example the term butenyl includes the isomeric groups but-1-enyl, but-2-enyl and but-3-enyl, etc.

In the above-mentioned alkenyl groups one or more hydrogen atoms may optionally be replaced by other groups. For example these alkenyl groups may be substituted by the halogen atoms fluorine, chlorine, bromine or iodine. The substituents fluorine or chlorine are preferred. The substituent fluorine is particularly preferred. If desired, all the hydrogen atoms of the alkenyl group may optionally also be replaced.

By alkynyl groups, as well as alkynyl groups which are a part of other groups, are meant branched and unbranched alkyl groups with 1 to 10 carbon atoms, preferably 1 to 6, particularly preferably 1 to 4 carbon atoms, which contain at least one carbon-carbon triple bond. Examples include: ethynyl, propynyl, butynyl, pentynyl, hexynyl, heptynyl, octynyl, nonynyl and decynyl. Unless stated otherwise, the above-mentioned terms propynyl, butynyl, pentynyl, hexynyl, heptynyl, octynyl, nonynyl and decynyl include all the possible isomeric forms. For example the term butynyl includes the isomeric groups but-1-ynyl, but-2-ynyl and but-3-ynyl, etc.

In the above-mentioned alkynyl groups one or more hydrogen atoms may optionally be replaced by other groups. For example these alkynyl groups may be substituted by the halogen atoms fluorine, chlorine, bromine or iodine. The substituents fluorine or chlorine are preferred. The substituent fluorine is particularly preferred. If desired, all the hydrogen atoms of the alkynyl group may optionally also be replaced.

The term aryl denotes an aromatic ring system with 6 to 18 carbon atoms, preferably 6 to 14 carbon atoms, preferably 6 or 10 carbon atoms, particularly preferably phenyl, which may optionally be substituted and may preferably carry one or more of the following substituents: OH, NO₂, CN, —OCHF₂, —OCF₃, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂—N(alkyl)₂, —NHSO₂-aryl, —N(alkyl)-SO₂-alkyl, —N(alkyl)-SO₂-aryl, —CO₂-alkyl, —SO₂-alkyl, —SO₂-aryl, —CONH(OH), —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, tetrazolyl, halogen, for example fluorine, chlorine, bromine or iodine, preferably fluorine or chlorine, particularly fluorine, C₁-C₁₀-alkyl, preferably C₁-C₅-alkyl, particularly preferably C₁-C₃-alkyl, most particularly preferably methyl or ethyl, —O—(C₁-C₃-alkyl), preferably methoxy or ethoxy, —COOH or —CONH₂.

By heteroaryl groups are meant 5- to 10-membered mono- or bicyclic heteroaryl rings, wherein one to three carbon atoms may be replaced in each case by a heteroatom selected from among oxygen, nitrogen or sulphur. Examples include furan, thiophene, pyrrole, pyrazole, imidazole, triazole, tetrazole, pyridine, pyridazine, pyrimidine, pyrazine, triazine, oxazole, isoxazole, thiazole, thiadiazole, oxadiazole, while each of the above-mentioned heterocycles may optionally also be annelated to a benzene ring, such as for example benzimidazole, and these heterocycles may optionally be substituted and may preferably carry one or more of the following substituents: OH, NO₂, CN, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂-aryl, —N(alkyl)-SO₂-alkyl, —N(alkyl)-SO₂-aryl, —CO₂-alkyl, —SO₂-alkyl, —SO₂-aryl, —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, —CONH₂, halogen, preferably fluorine or chlorine, C₁-C₁₀-alkyl, preferably C₁-C₅-alkyl, preferably C₁-C₃-alkyl, particularly preferably methyl or ethyl, —O—(C₁-C₃-alkyl), preferably methoxy or ethoxy, —COOH, —COOCH₃, —CONH₂, —SO-alkyl, —SO₂-alkyl, —SO₂H, —SO₃-alkyl or optionally substituted phenyl.

The term cycloalkyl groups denotes saturated or unsaturated cycloalkyl groups with 3 to 8 carbon atoms such as for example cyclopropyl, cyclobutyl, cyclopentyl, cyclo-pentenyl, cyclohexyl, cyclohexenyl, cycloheptyl or cyclooctyl, preferably cyclopropyl, cyclopentyl or cyclohexyl, while each of the above-mentioned cycloalkyl groups may optionally also carry one or more substituents or be annelated to a benzene ring.

By heterocycloalkyl or heterocyclyl groups are meant, unless otherwise described in the definitions, 5-, 6- or 7-membered, saturated or unsaturated heterocycles, which may contain nitrogen, oxygen or sulphur as heteroatoms, such as for example tetrahydrofuran, tetrahydrofuranone, γ-butyrolactone, α-pyran, γ-pyran, dioxolane, tetrahydropyran, dioxane, dihydrothiophene, thiolan, dithiolan, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, pyrazolidine, preferably pyrazolyl, pyrrolidinyl, piperidinyl, piperazinyl or tetrahydro-oxazinyl, while the heterocyclic group may optionally be substituted.

The compounds of the above general formula (I) which contain a group that can be cleaved in-vivo are so-called prodrugs, and compounds of general formula I which contain two groups that can be cleaved in-vivo are so-called double prodrugs.

By a group that can be converted in-vivo into a carboxy group is meant for example an ester of formula —CO₂R¹¹, wherein

R¹¹ denotes hydroxymethyl, alkenyl, alkynyl, aryl, heteroaryl, cycloalkenyl, hetero-cycloalkyl, C₁-C₃-alkoxycarbonyl, 1,3-dihydro-3-oxo-1-isobenzofuranol, —C(-alkyl)(-alkyl)-OC(O)-alkyl, —CHC(O)NH(-alkyl), —CHC(O)N(-alkyl)(-alkyl),
alkyl, preferably C₁-C₆-alkyl, particularly preferably methyl, ethyl, n-propyl, iso-propyl, n-butyl, n-pentyl or n-hexyl,
cycloalkyl, preferably C₁-C₆-cycloalkyl, particularly preferably cyclohexyl,
—(C₁-C₃-alkyl)-aryl, preferably (C₁-C₃-alkyl)-phenyl, particularly preferably benzyl,
—CHC(O)N(-alkyl)(-alkyl), preferably —CHC(O)N(—C₁-C₃-alkyl)(—C₁-C₃-alkyl), particularly preferably —CHC(O)N(CH₃)₂,
—CH(-alkyl)OC(O)-alkyl, preferably —CH(—CH₃)OC(O)(—C₁-C₆-alkyl), particularly preferably —CH(—CH₃)OC(O)-methyl, —CH(—CH₃)OC(O)-ethyl, —CH(—CH₃)OC(O)-n-propyl, —CH(—CH₃)OC(O)-n-butyl or —CH(—CH₃)OC(O)-t-butyl, or —CH₂OC(O)-alkyl, preferably —CH₂OC(O)(—C₁-C₆-alkyl), particularly preferably
—CH₂OC(O)-methyl, —CH₂OC(O)-ethyl, —CH₂OC(O)-n-propyl, —CH₂OC(O)-n-butyl or —CH₂OC(O)-t-butyl.

By a group that can be converted in-vivo into a sulphonamide or amino group is meant for example one of the following groups:

—OH, -formyl, —C(O)-alkyl, —C(O)-aryl, —C(O)-heteroaryl, —CH₂OC(O)-alkyl,

### —CH(-alkyl)OC(O)-alkyl, —C(-alkyl)(-alkyl)OC(O)-alkyl,

—CO₂-alkyl, preferably C₁-C₉-alkoxy-carbonyl, particularly preferably methoxycarbonyl, ethoxycarbonyl, n-propyloxycarbonyl, isopropyloxycarbonyl, n-butyloxycarbonyl, n-pentyloxycarbonyl, n-hexyloxycarbonyl, cyclohexyloxycarbonyl, n-heptyloxycarbonyl, n-octyloxycarbonyl or n-nonyloxycarbonyl,
—CO₂(—C₁-C₃-alkyl)-aryl, preferably —CO₂(—C₁-C₃-alkyl)-phenyl, particularly preferably benzyloxycarbonyl,
—C(O)-aryl, preferably benzoyl,
—C(O)-heteroaryl, preferably pyridinoyl or nicotinoyl or
—C(O)-alkyl, preferably —C(O)(—C₁-C₆-alkyl), particularly preferably 2-methylsulphonyl-ethoxycarbonyl, 2-(2-ethoxy)-ethoxycarbonyl.

The term halogen generally denotes fluorine, chlorine, bromine or iodine, preferably chlorine or fluorine, particularly preferably fluorine.

The compounds according to the invention may be in the form of the individual optical isomers, mixtures of the individual enantiomers, diastereomers or racemates, prodrugs, double prodrugs and in the form of the tautomers, salts, solvates and hydrates thereof as well as in the form of the free bases or the corresponding acid addition salts with pharmacologically acceptable acids—such as for example acid addition salts with hydrohalic acids, for example hydrochloric or hydrobromic acid, or organic acids, such as for example oxalic acid, fumaric acid, diglycolic acid, formic acid, malic acid, benzoic acid, benzenesulphonic acid, camphorsulphonic acid, acetic acid, ethanesulphonic acid, glutamic acid, maleic acid, mandelic acid, lactic acid, phosphoric acid, nitric acid, sulphuric acid, succinic acid, para-toluenesulphonic acid, trifluoroacetic acid, tartaric acid, citric acid or methanesulphonic acid.

Moreover, if the new compounds of formula I thus obtained contain a carboxy group or another acid group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

Moreover the compounds of general formula I obtained may be resolved into their enantiomers and/or diastereomers.

Thus, for example, the compounds of general formula I obtained which occur as racemates may be separated by methods known per se (cf. Allinger N. L. And Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

As has been found, the compounds of general formula (I) are characterised by their great versatility in the therapeutic field. Particular mention should be made of those applications in which the effects of beta-3-agonists, particularly selective beta-3-agonists play a part.

Such diseases include for example:

atherosclerosis, cholangitis, gall bladder disease, chronic cystitis, chronic bladder inflammation; chronic prostatitis, cystospasm, depression, duodenal ulcer, duodenitis, dysmenorrhoea, increased intraocular pressure and glaucoma, enteritis, oesophagitis, gastric ulcer, gastritis, gastrointestinal disorders caused by contraction(s) of the smooth muscle, gastrointestinal disorders incl. gastric ulcer, gastrointestinal ulceration, gastrointestinal ulcers, glaucoma, glucosuria, hyperanakinesia, hypercholesterolaemia, hyperglycaemia, hyperlipaemia, arterial hypertension, hypertriglyceridaemia, insulin resistance, intestinal ulceration or small bowel ulcers (incl. inflammatory bowel diseases, ulcerative colitis, Crohn's disease and proctitis=inflammation of the rectum), irritable colon and other diseases with decreased intestinal motility, depression, melancholy, pollacisuria, frequent urinary urgency, nervous neurogenic inflammation, neurogenic bladder dysfunction, neurogenic inflammation of the respiratory tract, neuropathic bladder dysfunction, nycturia, non-specific diarrhoea, dumping syndrome, obesity, fatness, pancreatitis, inflammation of the pancreas, stomach ulcers, prostate diseases such as benign prostatic hyperplasia, enlarged prostate, spasm, cramp, type 2 diabetes mellitus, irritable bladder or concrement of the lower urinary tract.

The following may also be mentioned: urge incontinence, stress incontinence, mixed incontinence, overactive bladder (OAB) in the forms of wet OAB or dry OAB, OAB with imperative need to urinate, with or without urge incontinence, with or without increased frequency of urination, with or without nocturnal urination, dysuria, nycturia, pollacisuria, build-up of residual urine. Of these indications, OAB with increased frequency of urination, with or without urge incontinence, with or without nocturnal urination, is preferred.

The compounds may also be used in cases of pain in the prostate or of the lower urogenital tract. The diseases in question include benign prostatic hyperplasiam (BPH), prostatitis, particularly chronic abacterial prostatitis, of neurogenic, muscular or bacterial origin, chronic pain syndrome of the pelvis, pelvic myoneuropathy, prostatodynia, LUTS (lower urinary tract symptoms), obstructive bladder emptying disorders (BOO) and/or prostatopathy.

The use according to the invention is directed not only to causative treatment of the above indications, but also to the treatment of the accompanying symptoms, particularly any related pain or problems of urine release, pain and discomfort in the region of the prostate or the lower urinary tract including the penis, pain during erection or ejaculation, pain on defecation, erectile disorders.

The beta-3 agonists according to the invention are particularly suitable for the treatment of obesity, insulin resistance, type 2 diabetes mellitus, urinary incontinence, irritable colon and other diseases with decreased intestinal motility or depression, particularly for the treatment of diabetes and obesity.

The activity of the beta-3 agonists can be determined for example in a lipolysis test. The test procedure may be carried out as follows:

Adipocytes were isolated from fatty tissue ex vivo by modifying a method according to Rodbell (Rodbell, M. Metabolism of isolated fat cells. I. Effects of hormones on glucose metabolism and lipolysis. J Biol Chem 239: 375-380.1964). The excised fatty tissue was cut into small pieces and mixed with 1 mg/ml collagenase in Krebs Ringer Buffer (KRB) containing 6 mM glucose and 2% albumin by gently shaking for 30-40 min at 37° C. The cells were filtered through a gauze, washed twice with KRB and in each case 50-150 g were centrifuged for 5 min. 10 μl of the centrifuged adipocytes were incubated with 90 μl of a compound according to the invention (agonist) at concentrations of between 10⁻¹⁵ to 10⁻⁴ M. The agonists were incubated over 40 min at 37° C. A varying release of glycerol into the medium indicated that the fat cell lipolysis had altered as a result of the addition of the agonist. Released glycerol was detected enzymatically with a Sigma kit (Triglyceride (GPO Trinder) Reagent A; Cat. # 337-40A), as described below.

Glycerol is phosphorylated by ATP via glycerol kinase. The resulting glycerol-1-phosphate is oxidised by glycerolphosphate oxidase to form dihydroxyacetone phosphate and hydrogen peroxide. Then a quinonimine dye is produced by the peroxidase-catalysed coupling of sodium-N-ethyl-N-(3-sulphopropyl)m-ansidine and 4-aminoantipyrine. The dye has an absorption peak at 540 nm. The absorption is directly proportional to the glycerol concentration in the samples.

The new compounds may be used for the prevention or short-term or long-term treatment of the above-mentioned diseases, and may also be used in conjunction with other active substances used for the same indications. These include, for example, antidiabetics, such as metformin, sulphonylureas (e.g. glibenclamid, tolbutamide, glimepiride), nateglinide, repaglinide, thiazolidinedione (e.g. rosiglitazone, pioglitazone), PPAR-gamma agonists (e.g. GI 262570), alpha-gluco-sidase inhibitors (e.g. acarbose, voglibose), alpha2 antagonists, insulin and insulin analogues, GLP-1 and GLP-1 analogues (e.g. Exendin-4) or amylin. Also, inhibitors of protein tyrosine phosphatase 1, substances which influence deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents, such as HMG-CoA-reductase inhibitors (e.g. simvastatin, atorvastatin), fibrates (e.g. bezafibrate, fenofibrate), nicotinic acid and its derivatives, cholesterol absorption inhibitors such as for example ezetimibe, bile acid-binding substances such as for example cholestyramine, HDL-raising compounds such as for example inhibitors of CETP or regulators of ABC1 or active substances for the treatment of obesity, such as e.g. sibutramine or tetrahydrolipostatin.

In particular, they may also be combined with drugs for treating high blood pressure such as e.g. all antagonists or ACE inhibitors, diuretics, β-blockers, and other modulators of the adrenergic system or combinations thereof. In addition, combinations with stimulators of the adrenergic system via alpha 1 and alpha 2 and also beta 1, beta 2 and beta 3 receptors are particularly suitable.

The compounds of general formula (I) may be used on their own or in conjunction with other active substances according to the invention, optionally also in conjunction with other pharmacologically active substances. Suitable preparations include for example tablets, capsules, suppositories, solutions, particularly solutions for injection (s.c., i.v., i.m.) and infusion, elixirs, emulsions or dispersible powders. The content of the pharmaceutically active compound(s) should be in the range from 0.1 to 90 wt. %, preferably 0.5 to 50 wt. % of the composition as a whole, i.e. in amounts which are sufficient to achieve the dosage range specified below. The specified doses may be taken several times a day, if necessary.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, optionally organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, preferably oral. For oral administration the tablets may, of course contain, apart from the above-mentioned carriers, additives such as sodium citrate, calcium carbonate and dicalcium phosphate together with various added substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation Examples which follow illustrate the present invention without restricting its scope:

### EXAMPLES OF PHARMACEUTICAL FORMULATIONS

A)

| Tablets | per tablet |
|---|---|
| active substance | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B)

| Tablets | per tablet |
|---|---|
| active substance | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

C)

| Ampoule solution | |
|---|---|
| active substance | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

### Component 1: N-(3-acetyl-phenyl)-benzenesulphonamide

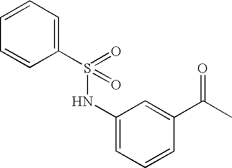

Summa, Vincenzo; Petrocchi, Alessia; Pace, Paola; Matassa, Victor G.; Francesco, Raffaele De; Altamura, Sergio; Tomei, Licia; Koch, Uwe; Neuner, Philippe; J. Med. Chem.; 47; 1; 2004; 14-17.

### Component 2: Synthesis of N-[3-(2-ethoxy-2-hydroxyacetyl)-phenyl]-benzenesulphonamide

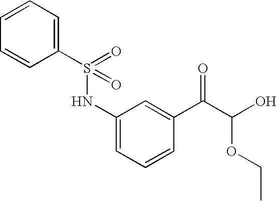

1 mL water, 1 g activated charcoal and 2.66 g (24 mmol) selenium dioxide was added to a solution of 1.65 g (6.00 mmol) N-(acetylphenyl)benzenesulphonamide in 10 mL dioxane. The reaction mixture was stirred for 4 days at 80° C. and then freed from the solvent using the rotary evaporator. The residue was dissolved in 30 mL ethanol and refluxed for 4 h. The reaction mixture was freed from the solvent using the rotary evaporator, dissolved in 100 mL ethyl acetate, washed with saturated, aqueous sodium hydrogen carbonate solution, dried on sodium sulphate and evaporated down using the rotary evaporator. 0.917 g (2.73 mmol, 46%) N-[3-(2-ethoxy-2-hydroxyacetyl)-phenyl]-benzenesulphonamide were obtained as a yellow solid. ESI MS [M+H]⁺=336; R_{f}=0.21 (silica gel, petroleum ether/ethyl acetate 1:1)

### Component 3: Synthesis of N-[3-(2-chloro-1-hydroxy-ethyl)-phenyl]-benzenesulphonamide (3 steps)

### Step 1: Synthesis of N-[3-(2-chloro-1-hydroxy-ethyl)-phenyl]-benzenesulphonamide

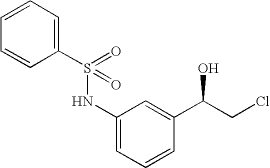

17.8 g (55.4 mmol) (−)-B-chlorodiisopinocamphenylboran [(−)-DIP-chloride] (dissolved in 20 mL tetrahydrofuran) were added dropwise at −30° C. to 5.20 g (16.8 mmol) N-[3-(2-chloro-acetyl)-phenyl]-benzenesulphonamide in tetrahydrofuran. The reaction mixture was stirred for 15 hours (h) at this temperature, then poured into ice-cooled, saturated, aqueous sodium hydrogen carbonate solution and extracted with ethyl acetate. The combined organic phases were washed successively with water and saturated aqueous sodium chloride solution, dried on magnesium sulphate and evaporated down using the rotary evaporator. The residue was purified by flash column chromatography [silica gel, petroleum ether/ethyl acetate (95:5->60:40)], to obtain 4.40 g (14.1 mmol, 84%) (R)—N-[3-(2-chloro-1-hydroxy-ethyl)-phenyl]-benzenesulphonamide.

ESI MS [(M−H)⁻]=310/12 (Cl); R_{f}=0.15 (silica gel, petroleum ether/ethyl acetate 2:1)]

### Step 2: Synthesis of N-[3-(2-Iodo-1-hydroxy-ethyl)-phenyl]-benzenesulphonamide

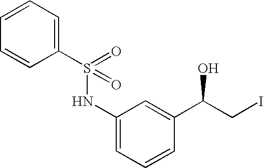

4.87 g (32.5 mmol) sodium iodide were added at ambient temperature to 9.65 g (31.0 mmol) N-[3-(2-chloro-1-hydroxy-ethyl)-phenyl]-benzenesulphonamide in 15 mL dimethylformamide. The reaction mixture was refluxed for 3 days and then evaporated down using the rotary evaporator. The residue was combined with 500 mL water and extracted with ethyl acetate. The combined organic phases were washed successively with 5% aqueous sodium thiosulphate solution, water and saturated aqueous sodium chloride solution, dried on sodium sulphate and evaporated down using the rotary evaporator. 13.3 g (31.0 mmol, quantitative) N-[3-(2-Iodo-1-hydroxy-ethyl)-phenyl]-benzenesulphonamide were obtained as a beige oil. ESI MS [M−H]-=402; R_{f}=0.45 (silica gel, toluene/ethyl acetate 7:3)

### Step 3: Synthesis of N-{3-[1-(tert-butyl-dimethyl-silanyloxy)-2-iodo-ethyl]-phenyl}benzenesulphonamide

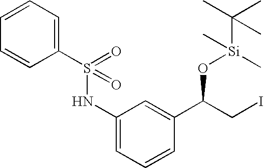

14.8 g (98.2 mmol) tert-butylchlorodimethylsilane (dissolved in 20 mL toluene) were added dropwise at 0° C. to 13.2 g (32.7 mmol) N-[3-(2-Iodo-1-hydroxy-ethyl)-phenyl]-benzenesulphonamide, 13.7 (196 mmol) imidazole and 0.04 g (0.33 mmol) 4-dimethylaminopyridine in 83 mL dimethylformamide over 30 minutes (min). The reaction mixture was stirred for 18 h at ambient temperature, combined with 4 mL methanol, stirred for 5 min at ambient temperature, combined with 800 mL water and extracted with ethyl acetate. The combined organic phases were washed successively with water and saturated aqueous sodium chloride solution, dried on magnesium sulphate and evaporated down using the rotary evaporator. 16.9 g (31.0 mmol, quantitative) N-{3-[1-(tert-butyl-dimethyl-silanyloxy)-2-iodo-ethyl]-phenyl}-benzenesulphonamide were obtained as a yellow oil. ESI MS [M−H]-=516; R_{f}=0.53 (silica gel, toluene/ethyl acetate 9:1)

### Component 4: tert-butyl (3-chloro-1,1-dimethyl-propyl)-carbamate (2 steps)

### Step 1: Synthesis of 3-chloro-1,1-dimethylpropylamine-hydrochloride

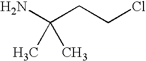

48.7 mL (668 mmol) thionyl chloride were slowly added dropwise at 0° C. to a solution of 53.0 g (514 mmol) 3-amino-3-methyl-butanol in 255 mL dichloromethane/dimethylformamide (50/1). After the addition had ended the reaction mixture was refluxed for 1 h and then stirred for 16 h at ambient temperature. The reaction mixture was evaporated down using the rotary evaporator and the residue was combined with 50 mL acetonitrile with stirring. It was filtered and the solid obtained was dried for 18 h at 45° C. 67.9 g (430 mmol, 84%) 3-chloro-1,1-dimethylpropylamine-hydrochloride were obtained as a colourless solid (60%). Further reacted in the next step.

ESI MS [M+H]⁺=122/124/Cl); R_{f}=0.52 (silica gel, dichloromethane/methanol/ammonia 90:10:1)

### Step 2: Synthesis of tert-butyl (3-chloro-1,1-dimethyl-propyl)-carbamate

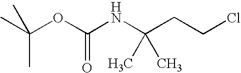

101 g (218 mmol) di-tert.-butyldicarbonate were added batchwise at ambient temperature to a solution of 48.8 g (309 mmol) 3-chloro-1,1-dimethylpropylamine-hydrochloride and 100 mL (718 mmol) triethylamine in 900 mL dichloromethane. After the addition had ended the reaction mixture was stirred for 4 d at RT. The reaction mixture was evaporated down using the rotary evaporator and the residue was taken up in 250 mL ethyl acetate and 400 mL water. The phases were separated and the aqueous phase was extracted with ethyl acetate. The combined organic phases were washed with water, dried on sodium sulphate and evaporated down using the rotary evaporator. 45.3 g (204 mmol, 66%) tert-butyl (3-chloro-1,1-dimethyl-propyl)-carbamate were obtained as a colourless oil.

ESI MS [M+H]⁺=222/224 (Cl); R_{f}=0.90 (silica gel, dichloromethane/methanol 9:1)

### Component 5: ethyl imidazo[4,5-c]pyridine-3-carboxylate

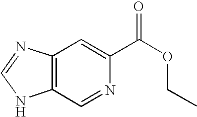

Ethyl imidazo[4,5-c]pyridine-3-carboxylate was prepared analogously to the synthesis of the methyl ester known from the literature: Guzman, Filadelfo; Cain, Michael; Larscheid, Paul; Hagen, Tim; Cook, James M.; et al.; J. Med. Chem.; 27; 5; 1984; 564-570.

ESI MS [M+H]⁺=192; R_{f}=0.33 (silica gel, dichloromethane/methanol 9:1)

### Component 6 and 7: ethyl 3-(3-amino-3-methyl-butyl)-3H-imidazo[4,5-c]pyridine-6-carboxylate and ethyl 1-(3-amino-3-methyl-butyl)-1H-imidazo[4,5-c]pyridine-6-carboxylate

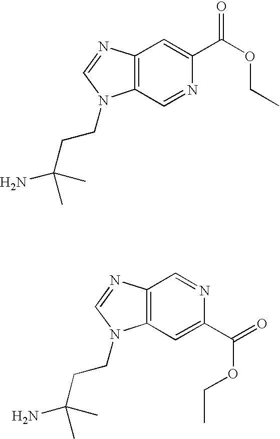

1.13 g (28.2 mmol) sodium hydride (60% in mineral oil) were added to 4.00 g (20.9 mmol) ethyl imidazo[4,5-c]pyridine-3-carboxylate in 5 mL of 1,3-dimethyl-3,4,5,6-tetrahydro-2(1H)-pyrimidone at 5° C. and stirred for 30 min at ambient temperature. The reaction mixture was then combined with 6.96 g (31.4 mmol) tert-butyl (3-chloro-1,1-dimethyl-propyl)-carbamate and 0.774 g (2.10 mmol) tetrabutylammonium iodide and stirred for 72 h at 60° C. The reaction mixture was poured into ice water and extracted with ethyl acetate. The combined organic phases were washed successively with water and saturated, aqueous sodium chloride solution, dried on magnesium sulphate and evaporated down using the rotary evaporator.

The residue was dissolved in 30 mL dichloromethane and at ambient temperature combined with 2 mL trifluoroacetic acid. The reaction mixture was stirred for 1.5 h at ambient temperature and then evaporated down using the rotary evaporator. The residue was purified by reversed-phase flash column chromatography {Varian Microsorb C18-Reversed phase [acetonitrile (0.1% trifluoroacetic acid)/water (0.13% trifluoroacetic acid)=10:90->100:0]}, to obtain 1.80 g (6.51 mmol, 21%) ethyl 3-(3-amino-3-methyl-butyl)-3H-imidazo[4,5-c]pyridine-6-carboxylate and 1.20 g (4.33 mmol, 14%) ethyl 1-(3-amino-3-methyl-butyl)-1H-imidazo[4,5-c]pyridine-6-carboxylate as colourless oils.

### Component 7: ethyl 3-(3-amino-3-methyl-butyl)-3H-imidazo[4,5-b]pyridine-5-carboxylate (4 steps)

### Step 1: Synthesis of (6-chloro-3-nitro-pyridin-2-yl)-(3-methyl-3-nitro-butyl)-amine

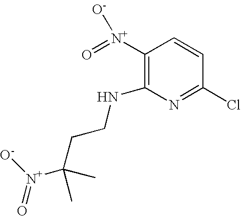

5.00 g (25.9 mmol) 2,6-dichloro-3-nitropyridine were added at ambient temperature to 4.37 g (25.9 mmol) 3-methyl-3-nitro-butylamine-hydrochloride and 7.29 mL (101 mmol) triethylamine in 150 mL dichloromethane. The reaction mixture was stirred for 20 h at ambient temperature and then evaporated down using the rotary evaporator. The residue was mixed with water and extracted with ethyl acetate. The combined organic phases were washed successively with water and saturated aqueous sodium chloride solution, dried on sodium sulphate and evaporated down using the rotary evaporator. 7.45 g (25.8 mmol, quantitative) (6-chloro-3-nitro-pyridin-2-yl)-(3-methyl-3-nitro-butyl)-amine were obtained.

ESI MS [M+H]⁺=288/290 (Cl); R_{f}=0.69 (silica gel, petroleum ether/ethyl acetate 4:1)

### Step 2: Synthesis of 6-(3-methyl-3-nitro-butylamino)-5-nitro-pyridine-2-carbonitrile

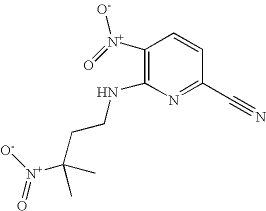

6.3 g (21.8 mmol) (6-chloro-3-nitro-pyridin-2-yl)-(3-methyl-3-nitro-butyl)-amine and 3.91 g (43.6 mmol) copper(I)cyanide were stirred in 20 mL 1-methyl-2-pyrrolidinone for 24 h at 160° C. The reaction mixture was mixed with water, whereupon a precipitate was formed. The reaction mixture was filtered and the filter cake was washed with ethyl acetate. The filtrate was evaporated down using the rotary evaporator. The residue was purified by flash column chromatography [silica gel, dichloromethane/methanol (95/5)]. 2.9 g (10.4 mmol, 48%) 6-(3-methyl-3-nitro-butyl-amino)-5-nitro-pyridine-2-carbonitrile were obtained.

ESI MS [M+H]⁺=280; R_{f}=0.53 (silica gel, petroleum ether/ethyl acetate 4:1)

### Step 3: Synthesis of ethyl 6-(3-methyl-3-nitro-butylamino)-5-nitro-pyridine-2-carboxylate

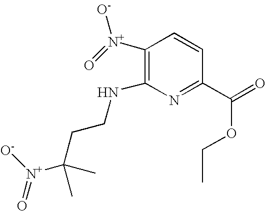

2.8 g (10.3 mmol) 6-(3-methyl-3-nitro-butylamino)-5-nitro-pyridine-2-carbonitrile and 90 mL (455 mmol) ethanolic hydrogen chloride solution (approx. 5M) were stirred for 20 h at 50° C. The reaction mixture was evaporated down using the rotary evaporator. The residue was mixed with water and extracted with dichloromethane. The combined organic phases were dried on sodium sulphate and evaporated down using the rotary evaporator. 3.4 g (10.4 mmol, quantitative) ethyl 6-(3-methyl-3-nitro-butylamino)-5-nitro-pyridine-2-carboxylate were obtained.

ESI MS [M+H]⁺=327; R_{f}=0.58 (silica gel, petroleum ether/ethyl acetate 4:1)

### Step 4: Synthesis of ethyl 3-(3-amino-3-methyl-butyl)-3H-imidazo[4,5-b]pyridine-5-carboxylate

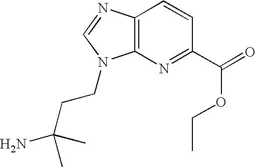

3.4 g (10.4 mmol) ethyl 6-(3-methyl-3-nitro-butylamino)-5-nitro-pyridine-2-carboxylate, 0.30 g palladium on charcoal and 20 mL methanol in 70 mL ethyl acetate were shaken for 24 h at ambient temperature in an autoclave at 3 bar hydrogen atmosphere. The reaction mixture was filtered and the filtrate was evaporated down using the rotary evaporator. The residue was taken up in 70 mL ethanol, combined with 0.30 g palladium on charcoal and shaken for 18 h at ambient temperature in an autoclave at 3 bar hydrogen atmosphere. The reaction mixture was filtered and the filtrate was evaporated down using the rotary evaporator. The residue was combined with 75 mL formic acid and refluxed for 3 h. The reaction mixture was evaporated down using the rotary evaporator and the residue was purified by flash column chromatography [silica gel, dichloromethane/methanol/ammonia (99/0/1->70/30/1)]. 2.00 g (7.24 mmol, 69%) ethyl 3-(3-amino-3-methyl-butyl)-3H-imidazo[4,5-b]pyridine-5-carboxylate were obtained.

ESI MS [M+H]⁺=277; R_{f}=0.28 (silica gel, dichloromethane:methanol/ammonia 80:20:0.1)

### Example 1

### Ethyl (R)-3-{3-[2-(3-benzenesulphonylamino-phenyl)-2-hydroxy-ethylamino]-3-methyl-butyl}-3H-imidazo[4,5-b]pyridine-5-carboxylate

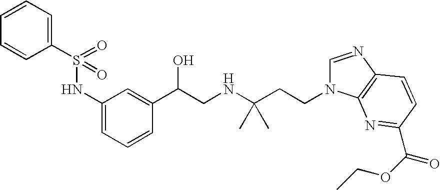

1.19 g (2.29 mmol) N-{3-[1-(tert-butyl-dimethyl-silanyloxy)-2-iodo-ethyl]-phenyl}benzenesulphonamide, 0.70 g (2.52 mmol) ethyl 3-(3-amino-3-methyl-butyl)-3H-imidazo[4,5-b]pyridine-5-carboxylate and 0.48 g (3.43 mmol) potassium carbonate in 6.87 mL N,N-dimethylacetamide were stirred for 75 min at 120° C. The reaction mixture was poured into water and extracted with ethyl acetate. The combined organic phases were washed successively with water and saturated aqueous sodium chloride solution, dried on magnesium sulphate and evaporated down using the rotary evaporator. The residue was purified by flash column chromatography [dichloromethane/methanol (100/0->90/10)]. 0.09 g silylated intermediate product were obtained. This was dissolved in 1.0 mL methanol and 0.5 mL glacial acetic acid and 50 mg ammonium fluoride were added. The reaction mixture was stirred for 18 h at ambient temperature and then evaporated down using the rotary evaporator. The residue was purified by reversed-phase flash column chromatography {Varian Microsorb C18-Reversed phase [acetonitrile (0.1% trifluoroacetic acid)/water (0.13% trifluoroacetic acid)=10:90->100:0]}. 0.07 g (0.01 mmol, 4.0%) ethyl 3-{3-[2-(3-benzenesulphonylamino-phenyl)-2-hydroxy-ethylamino]-3-methyl-butyl}-3H-imidazo-[4,5-b]pyridine-5-carboxylate trifluoroacetate were obtained as a colourless solid.

ESI MS [M+H]⁺=552; R_{f}=0.36 (silica gel, dichloromethane/methanol 9:1)]

### Example 2

### 3-{3-[2-(3-benzenesulphonylamino-phenyl)-2-hydroxy-ethylamino]-3-methyl-butyl}-3H-imidazo[4,5-b]pyridine-5-carboxylic acid

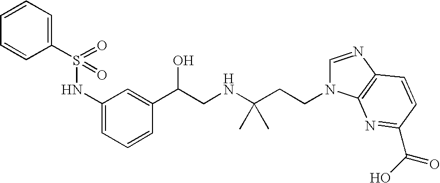

0.040 g (0.073 mmol) ethyl 3-{3-[2-(3-benzenesulphonylamino-phenyl)-2-hydroxy-ethylamino]-3-methyl-butyl}-3H-imidazo[4,5-b]pyridine-5-carboxylate were dissolved in 0.5 mL ethanol and 1 mL lithium hydroxide solution (2M in water) were added. The reaction mixture was stirred for 14 h at ambient temperature and then evaporated down using the rotary evaporator. The residue was purified by reversed-phase flash column chromatography {Varian Microsorb C18-Reversed phase [acetonitrile (0.1% trifluoroacetic acid)/water (0.13% trifluoroacetic acid)=10:90->100:0]}, and 0.033 g (0.052 mmol, 71%) 3-{3-[2-(3-benzenesulphonylamino-phenyl)-2-hydroxy-ethylamino]-3-methyl-butyl}-3H-imidazo[4,5-b]pyridine-5-carboxylic acid trifluoroacetate was obtained as a colourless solid.

ESI MS [M+H]⁺=552; R_{f}=0.28 (silica gel, dichloromethane:methanol 90:10)

### Example 3

### Ethyl 1-{3-[2-(3-benzenesulphonylamino-phenyl)-2-hydroxy-ethylamino]-3-methyl-butyl}-1H-imidazo[4,5-c]pyridine-6-carboxylate

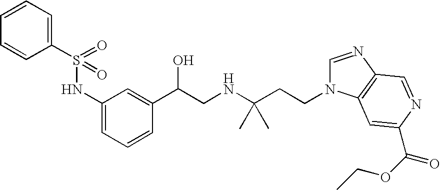

0.300 g (0.895 mmol) N-[3-(2-ethoxy-2-hydroxy-acetyl)-phenyl]-benzenesulphonamide and 0.370 g (1.34 mmol) ethyl 1-(3-tert-butoxycarbonylamino-3-methyl-butyl)-1H-imidazo[4,5-c]pyridine-6-carboxylate were dissolved in 8 mL ethanol and the pH value of the reaction mixture was adjusted with triethylamine to 8-9. The reaction mixture was refluxed for 18 h, then cooled to 0° C. and 0.130 g (3.44 mmol) sodium borohydride were added. The mixture was stirred for a further 2 h at ambient temperature and then poured into saturated aqueous sodium carbonate solution. The phases were separated and the aqueous phase was extracted with ethyl acetate. The combined organic phases were dried on sodium sulphate and evaporated down using the rotary evaporator. The residue was purified by flash column chromatography [silica gel, dichloromethane/methanol/ammonia (100/0/0->85/15/0.1)]. 0.120 g (0.218 mmol, 24%) ethyl 1-{3-[2-(3-benzenesulphonylamino-phenyl)-2-hydroxy-ethylamino]-3-methyl-butyl}-1H-imidazo[4,5-c]pyridine-6-carboxylate was obtained as a colourless solid.

ESI MS [M+H]⁺=524; R_{f}=0.37 (PR-18 F254, acetonitrile/water/glacial acetic acid 35:65:0.2)

### Example 4

### 1-{3-[2-(3-benzenesulphonylamino-phenyl)-2-hydroxy-ethylamino]-3-methyl-butyl}-1H-imidazo[4,5-c]pyridine-6-carboxylic acid trifluoroacetate

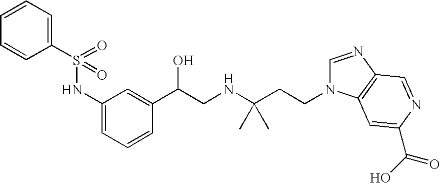

0.110 g (0.199 mmol) ethyl 1-{3-[2-(3-benzenesulphonylamino-phenyl)-2-hydroxy-ethylamino]-3-methyl-butyl}-1H-imidazo[4,5-c]pyridine-6-carboxylate were dissolved in 1 mL ethanol and 1 mL lithium hydroxide solution (3M in water) were added. The reaction mixture was stirred for 2 h at ambient temperature, then neutralised with 4N hydrochloric acid and purified by reversed-phase flash column chromatography {Varian Microsorb C18-Reversed phase [acetonitrile (0.1% trifluoroacetic acid)/water (0.13% trifluoroacetic acid)=10:90->100:0]}. 0.085 g (0.133 mmol, 67%) 1-{3-[2-(3-benzenesulphonylamino-phenyl)-2-hydroxy-ethylamino]-3-methyl-butyl}-1H-imidazo[4,5-c]pyridine-6-carboxylic acid trifluoroacetate was obtained as a colourless solid.

ESI MS [M+H]⁺=524; R_{f}=0.5 (silica gel, dichloromethane:methanol/ammonia 80:20:0.1)

### Example 5

### Ethyl 1-{3-[2-(3-benzenesulphonylamino-phenyl)-2-hydroxy-ethylamino]-3-methyl-butyl}-1H-imidazo[4,5-b]pyridine-5-carboxylate

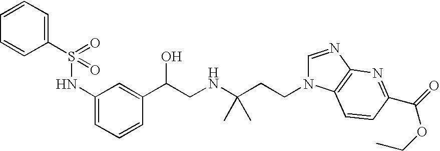

0.030 g (0.047 mmol) 1-{3-[2-(3-benzenesulphonylamino-phenyl)-2-hydroxy-ethyl-amino]-3-methyl-butyl}-1H-imidazo[4,5-b]pyridine-5-carboxylic acid in 3 mL ethanol were combined with 1 mL saturated ethanolic hydrochloric acid solution and refluxed for 3 h. The reaction mixture was poured into 50 mL saturated aqueous potassium carbonate solution and extracted with ethyl acetate. The combined organic phases were dried on sodium sulphate and evaporated down using the rotary evaporator. The residue was purified by reversed-phase flash column chromatography {Varian Microsorb C18-Reversed phase [acetonitrile (0.1% trifluoroacetic acid)/water (0.13% trifluoroacetic acid)=10:90->100:0]}. 0.028 g (0.042 mmol, 89%) ethyl 1-{3-[2-(3-benzenesulphonylamino-phenyl)-2-hydroxy-ethylamino]-3-methyl-butyl}-1H-imidazo-[4,5-b]pyridine-5-carboxylate trifluoroacetate was obtained as a colourless solid.

ESI MS [M+H]⁺=552; R_{f}=0.33 (silica gel, dichloromethane:methanol/ammonia 90:10:0.1)

### Example 6

### 1-{3-[2-(3-benzenesulphonylamino-phenyl)-2-hydroxy-ethylamino]-3-methyl-butyl}-1H-imidazo[4,5-b]pyridine-5-carboxylic acid (2 steps)

### Step 1: methyl 1-{3-[2-(3-benzenesulphonylamino-phenyl)-2-hydroxy-ethylamino]-3-methyl-butyl}-1H-imidazo[4,5-b]pyridine-5-carboxylate

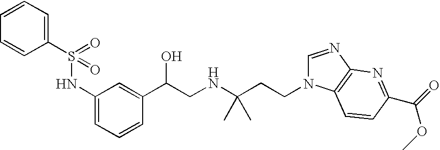

0.187 g (0.559 mmol) N-[3-(2-ethoxy-2-hydroxy-acetyl)-phenyl]-benzenesulphonamide and 0.220 g (0.839 mmol) ethyl 3-(3-amino-3-methyl-butyl)-3H-imidazo[4,5-b]pyridine-5-carboxylate were dissolved in 5 mL methanol and the pH value of the reaction mixture was adjusted to 8-9 with triethylamine. The reaction mixture was refluxed for 18 h, then cooled to 0° C., and 0.175 g (4.637 mmol) sodium borohydride were added. The mixture was stirred for a further 2 h at ambient temperature and then poured into saturated aqueous sodium carbonate solution. The phases were separated and the aqueous phase was extracted with ethyl acetate. The combined organic phases were dried on sodium sulphate and evaporated down using the rotary evaporator. The residue was purified by flash column chromatography [silica gel, dichloromethane/methanol/ammonia (100/0/0->85/15/0.1)]. 0.060 g (0.112 mmol, 20%) methyl 1-{3-[2-(3-benzenesulphonylamino-phenyl)-2-hydroxy-ethylamino]-3-methyl-butyl}-1H-imidazo[4,5-b]pyridine-5-carboxylate was obtained as a colourless solid.

ESI MS [M+H]⁺=538; R_{f}=0.27 (silica gel, dichloromethane:methanol/ammonia 90:10:0.1)

### Step 2: 1-{3-[2-(3-benzenesulphonylamino-phenyl)-2-hydroxy-ethylamino]-3-methyl-butyl}-1H-imidazo[4,5-b]pyridine-5-carboxylic acid

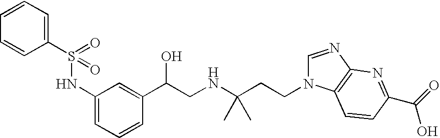

0.050 g (0.093 mmol) methyl 1-{3-[2-(3-benzenesulphonylamino-phenyl)-2-hydroxy-ethylamino]-3-methyl-butyl}-1H-imidazo[4,5-b]pyridine-5-carboxylate were dissolved in 1.5 mL methanol and 2 mL lithium hydroxide solution (3M in water) were added. The reaction mixture was stirred for 2 h at ambient temperature, then neutralised with 4N hydrochloric acid and purified by reversed-phase flash column chromatography {Varian Microsorb C18-Reversed phase [acetonitrile (0.1% trifluoroacetic acid)/water (0.13% trifluoroacetic acid)=10:90->100:0]}. 0.059 g (0.092 mmol, 99%) 1-{3-[2-(3-benzenesulphonylamino-phenyl)-2-hydroxy-ethylamino]-3-methyl-butyl}-1H-imidazo[4,5-b]pyridine-5-carboxylic acid trifluoroacetate was obtained as a colourless solid. ESI MS [M+H]⁺=524; R_{f}=0.5 (silica gel, dichloromethane/methanol/ammonia 80:20:0.1)

### Example 7

### Ethyl 3-{3-[2-(3-benzenesulphonylamino-phenyl)-2-hydroxy-ethylamino]-3-methyl-butyl}-3H-imidazo[4,5-c]pyridine-6-carboxylate

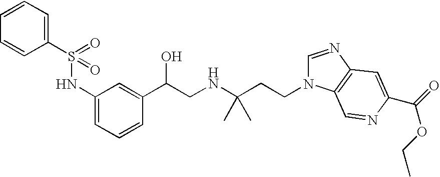

0.194 g (0.579 mmol) N-[3-(2-ethoxy-2-hydroxy-acetyl)-phenyl]-benzenesulphonamide and 0.600 g (0.869 mmol) ethyl 3-(3-tert-butoxycarbonylamino-3-methyl-butyl)-3H-imidazo[4,5-c]pyridine-6-carboxylate were refluxed in 5 mL ethanol for 18 h. The reaction mixture was cooled to 0° C. and then 0.175 g (4.64 mmol) sodium borohydride were added. The mixture was stirred for a further 2 h at ambient temperature and then poured into saturated aqueous sodium carbonate solution. The phases were separated and the aqueous phase was extracted with ethyl acetate. The combined organic phases were dried on sodium sulphate and evaporated down using the rotary evaporator. The residue was purified by flash column chromatography [silica gel, dichloromethane/methanol/ammonia (100/0/0->85/15/0.1)]. 0.060 g (0.109 mmol, 19%) ethyl 3-{3-[2-(3-benzenesulphonylamino-phenyl)-2-hydroxy-ethylamino]-3-methyl-butyl}-3H-imidazo[4,5-c]pyridine-6-carboxylate was obtained as a colourless solid.

ESI MS [M+H]⁺=552; R_{f}=0.33 (silica gel, dichloromethane:methanol/ammonia 90:10:0.1)

### Example 8

### 3-{3-[2-(3-benzenesulphonylamino-phenyl)-2-hydroxy-ethylamino]-3-methyl-butyl}-3H-imidazo[4,5-c]pyridine-6-carboxylic acid trifluoroacetate

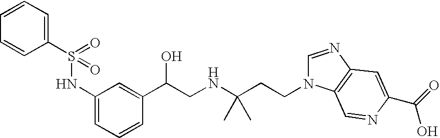

0.050 g (0.091 mmol) ethyl 3-{3-[2-(3-benzenesulphonylamino-phenyl)-2-hydroxy-ethylamino]-3-methyl-butyl}-3H-imidazo[4,5-c]pyridine-6-carboxylate were dissolved in 1.5 mL ethanol and 2 mL lithium hydroxide solution (3M in water) were added. The reaction mixture was stirred for 2 h at ambient temperature, then neutralised with 4N hydrochloric acid and purified by reversed-phase flash column chromatography {Varian Microsorb C18-Reversed phase [acetonitrile (0.1% trifluoroacetic acid)/water (0.13% trifluoroacetic acid)=10:90->100:0]}. 0.055 g (0.086 mmol, 95%) 3-{3-[2-(3-benzenesulphonylamino-phenyl)-2-hydroxy-ethylamino]-3-methyl-butyl}-3H-imidazo[4,5-c]pyridine-6-carboxylic acid trifluoroacetate was obtained as a colourless solid. ESI MS [M+H]⁺=524; R_{f}=0.5 (silica gel, dichloromethane:methanol/ammonia 80:20:0.1)

1: A compound of the formula 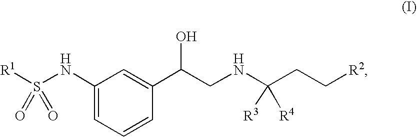
wherein
R¹ denotes a C₁₋₄-alkyl, thienyl, pyridyl or phenyl group,
wherein the phenyl group may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different,
R² denotes a benzimidazolyl group wherein a methyne group in the benzyl moiety is replaced by a nitrogen atom and
which may additionally be substituted by a fluorine, chlorine or bromine atom, a cyano group or a C₁₋₃-alkyl, carboxy, C₁₋₄-alkyloxy-carbonyl or amino group,
and R³ and R⁴, which may be identical or different, each represent a C₁₋₃-alkyl group, while the alkyl groups contained in the above-mentioned groups may be straight-chain or branched,
or a salt thereof.

2: A compound of the formula (I) according to claim 1, wherein
R¹ denotes a phenyl group,
R² denotes a benzimidazol-1-yl group wherein a methyne group in the benzyl moiety is replaced by a nitrogen atom and
which is additionally substituted by a carboxy or C₁₋₄-alkyloxy-carbonyl group,
and R³ and R⁴ in each case represent a methyl group,
or a tautomer or salt thereof.

3: A compound according to claim 1 wherein the compound is the (R)-enantiomer of formula

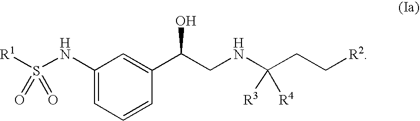

4: A compound according to claim 1 wherein the compound is the (S)-enantiomer of formula

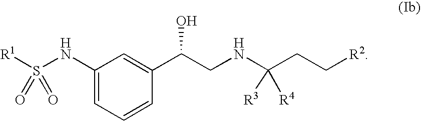

5: A compound according to claim 1 selected from the group consisting of:
ethyl 3-{3-[(R)-2-(3-benzenesulphonylamino-phenyl)-2-hydroxy-ethylamino]-3-methyl-butyl}-3H-imidazo[4,5-b]pyridine-5-carboxylate;
3-{3-[(R)-2-(3-benzenesulphonylamino-phenyl)-2-hydroxy-ethylamino]-3-methyl-butyl}-3H-imidazo[4,5-b]pyridine-5-carboxylic acid;
ethyl 1-{3-[2-(3-benzenesulphonylamino-phenyl)-2-hydroxy-ethylamino]-3-methyl-butyl}-1H-imidazo[4,5-c]pyridine-6-carboxylate;
1-{3-[2-(3-benzenesulphonylamino-phenyl)-2-hydroxy-ethylamino]-3-methyl-butyl}-1H-imidazo[4,5-c]pyridine-6-carboxylic acid;
ethyl 1-{3-[2-(3-benzenesulphonylamino-phenyl)-2-hydroxy-ethylamino]-3-methyl-butyl}-1H-imidazo[4,5-b]pyridine-5-carboxylate;
1-{3-[2-(3-benzenesulphonylamino-phenyl)-2-hydroxy-ethylamino]-3-methyl-butyl}-1H-imidazo[4,5-b]pyridine-5-carboxylic acid;
ethyl 3-{3-[(R)-2-(3-benzenesulphonylamino-phenyl)-2-hydroxy-ethylamino]-3-methyl-butyl}-3H-imidazo[4,5-c]pyridine-6-carboxylate; and
3-{3-[2-(3-benzenesulphonylamino-phenyl)-2-hydroxy-ethylamino]-3-methyl-butyl}-3H-imidazo[4,5-c]pyridine-6-carboxylic acid;
or an enantiomer or salt thereof.

6: A physiologically acceptable salt of a compound according to claim 1, 2, 3, 4 or 5.

7-10. (canceled)

11: A pharmaceutical composition comprising a compound according to claim 1, 2, 3, 4 or 5 or a pharmaceutically acceptable salt thereof and a carrier or excipient.

12-13. (canceled)

14: A method for the treatment of obesity or type II diabetes which comprises administering to a host suffering from the same a therapeutically effect amount of a compound according to claim 1, 2, 3, 4 or 5 or a pharmaceutically acceptable salt thereof.
